(19) United States
(12) Patent Application Publication
(10) Pub. No.: US 2007/0189980 A1
(43) Pub. Date: Aug. 16, 2007

(54) Compositions and methods for treating alopecia

(76) Inventors: Jie Zhang, Salt Lake City, UT (US); Kevin S. Wamer, West Jordan, UT (US); Sanjay Sharma, Sandy, UT (US)

Correspondence Address: THORPE NORTH & WESTERN, LLP., 8180 SOUTH 700 EAST, SUITE 350, SANDY, UT, 84070, US

(21) Appl. No.: 11/640,135

(22) Filed: Dec. 14, 2006

Related U.S. Application Data
(63) Continuation-in-part of application No. 11/146,917, filed on Jun. 6, 2005.
(60) Provisional application No. 60/750,637, filed on Dec. 14, 2005.
(60) Provisional application No. 60/577,536, filed on Jun. 7, 2004.

(51) Int. Cl. A61K 31/573; A61K 31/513; A61K 31/473; A61K 9/00

(52) U.S. Cl. 424/47; 514/171; 514/269; 514/284; 514/559

(57) ABSTRACT

The present invention is drawn to adhesive solidifying formulations, methods of drug delivery, and solidified layers for topical delivery of drugs for treating alopecia. The formulation can include a drug for treating alopecia, a solvent vehicle, and a solidifying agent. The solvent vehicle can include a volatile solvent system having one or more volatile solvent, and a non-volatile solvent system having one or more non-volatile solvent which is capable of facilitating the delivery of the drug at therapeutically effective rates over a sustained period of time. The formulation can have a viscosity suitable for application to a skin surface as a layer prior to evaporation of the volatile solvents system. When applied to the skin, the formulation can form a solidified layer after at least a portion of the volatile solvent system is evaporated.

This application claims the benefit of U.S. Provisional Application No. 60/750,637 filed on Dec. 14, 2005 and is a continuation-in-part of U.S. application Ser. No. 11/146,917 filed on Jun. 6, 2005, which claims the benefit of U.S. Provisional Application No. 60/577,536 filed on Jun. 7, 2004, each of which is incorporated herein by reference.

### FIELD OF THE INVENTION

The present invention relates generally to systems developed for dermal delivery of drugs for treating alopecia (hair loss). More particularly, the present invention relates to adhesive solidifying formulations having a viscosity suitable for application to a skin surface, and which form a sustained drug-delivering adhesive solidified layer on the skin.

### BACKGROUND OF THE INVENTION

Alopecia affects millions of people across the world. Although the cause and extent of the alopecia can differ from individual to individual, the ultimate outcome of hair loss is universal. Alopecia can affect both men and women and can be found in most races and ethnic groups throughout the world. Many attempts have been made in order to reverse or control the effects of alopecia, the most common involving the topical application of drug. Traditionally these drugs have been delivered in semisolid or injectable formulations. Semisolid formulations are available in a few different forms, including ointments, creams, foams, pastes, gels, or lotions and are applied topically to the skin While semisolid formulations are widely used to deliver drugs into and through the skin, they have significant limitations. For example, most semisolid formulations usually contain solvent(s), such as water and ethanol, which are volatile and thus evaporate shortly after application. The evaporation of such solvents can cause a significant decrease or even termination of dermal drug delivery, which may not be desirable in many cases. Additionally, semisolid formulations are often “rubbed into” the skin, which does not necessarily mean the drug formulation is actually delivered into the skin. Instead, this phrase often means that a very thin layer of the drug formulation is applied onto the surface of the skin. Such thin layers of traditional semisolid formulations applied to the skin may not contain sufficient quantity of active drug to achieve sustained delivery over long periods of time. Additionally, traditional semisolid formulations are often subject to unintentional removal due to contact with objects such as clothing, which may compromise the sustained delivery and/or undesirably soil clothing. Administration of injectable formulations often requires the assistance of a professional and is typically painful.

In view of the shortcomings of many of the current delivery systems for treating alopecia, it would be desirable to provide systems, formulations, and/or methods that can i) provide sustained drug delivery over long periods of time; ii) are not vulnerable to unintentional removal by contact with clothing, other objects, or people for the duration of the application time; iii) can be applied to a skin area subject to stretching and expansion without causing discomfort or poor contact to skin; and/or iv) can be easily removed after application and use.

### SUMMARY OF THE INVENTION

Although film-forming technologies have been used in cosmetic and pharmaceutical preparations, typically, the solvents used in such systems do not last very long, and thus, are not optimal for sustained-release applications.

In accordance with this, it would be advantageous to provide dermal delivery formulations, systems, and/or methods in the form of adhesive compositions or formulations having a viscosity suitable for application to the skin surface as a layer and which form a drug-delivering solidified layer on the skin that is optionally peelable or otherwise easily removable after use. As such, an adhesive solidifying formulation for dermal delivery of a drug can comprise a drug capable of stimulating hair growth when delivered to a hair follicle of a human skin area, a solvent vehicle, and a solidifying agent. The solvent vehicle can comprise a volatile solvent system including at least one volatile solvent and a non-volatile solvent system including at least one non-volatile solvent. The non-volatile solvent system can facilitate the delivery of the drug at a therapeutically effective rate over a sustained period of time, even after the volatile solvent system is substantially evaporated. The formulation can have viscosity suitable for application to the skin surface as a layer prior to evaporation of at least one volatile solvent, and can further be formulated such that when applied to the skin surface as a layer, the formulation forms a solidified layer after at least a portion of the volatile solvent system is evaporated. Sustained drug delivery from the solidified layer can also occur.

In an alternative embodiment, a method of treating alopecia can comprise applying a layer of an adhesive formulation to a skin surface suffering from alopecia (hair loss). The formulation can comprise a drug capable of stimulating hair growth when delivered to a hair follicle of a subject suffering from alopecia, a solvent vehicle, and a solidifying agent. The solvent vehicle can comprise a volatile solvent system including at least one volatile solvent, and a non-volatile solvent system including at least one non-volatile solvent, wherein the non-volatile solvent system is preferably capable of facilitating topical delivery of the drug at a therapeutically effective rate over a sustained period of time. The formulation can have a viscosity suitable for application and adhesion to the skin surface prior to evaporation of the volatile solvent system. Other steps include solidifying the formulation to form a solidified layer on the skin surface by at least partial evaporation of the volatile solvent system; and topically delivering the drug from the solidified layer to the skin at therapeutically effective rates over a sustained period of time.

In another embodiment, a solidified layer for delivering a drug for treating alopecia can comprise a drug capable of stimulating hair growth when delivered to a hair follicle of a subject suffering from alopecia, a non-volatile solvent system including at least one non-volatile solvent, wherein the non-volatile solvent system is capable of facilitating the delivery of the drug at therapeutically effective rates over a sustained period of time, and a solidifying agent. The solidified layer can have sufficient flexibility and adhesion to the skin surface so that it can maintain good contact with the skin surface to which it was originally applied for at least most of the intended duration of the application.

In another embodiment, a formulation for treating a subject suffering from alopecia can comprise a drug, a solvent vehicle, and a solidifying agent. The drug can include a member selected from the group consisting of clobetasol propionate, clobetasol, derivatives thereof, and combinations thereof. The solvent vehicle can comprise a volatile solvent system including at least one volatile solvent, and a non-volatile solvent system comprising at least one solvent selected from the group consisting of propylene glycol, glycerol, and combinations thereof, and at least one solvent selected from the group consisting of isostearic acid, oleic acid, and combinations thereof. The solidifying agent can include a member selected from the group consisting of polyvinyl alcohol, fish gelatin, gluten, casein, zein, and combinations thereof. The formulation can have a viscosity suitable for application and adhesion to a skin surface prior to evaporation of the volatile solvent system, and, after being applied to a skin surface as a layer, can form a solidified, coherent, flexible, and continuous layer after at least partial evaporation of the volatile solvent system. The continues to be topically delivered at the therapeutically effective rate after the volatile solvent system is at least substantially all evaporated.

In another embodiment, a method for treating alopecia can comprise applying to a skin area of a subject suffering from alopecia a 0.01 mm to 2 mm thick layer of an adhesive solidifying formulation. The formulation can comprise a drug including at least one member selected from the group consisting of clobetasol propionate, clobetasol, and combinations thereof, a volatile solvent system including at least one volatile solvent. Other ingredients can comprise a non-volatile solvent system including at least one solvent selected from the group consisting of propylene glycol, glycerol, and combinations thereof, and at least one solvent selected from the group consisting of isostearic acid, oleic acid, and combinations thereof. A solidifying agent can also be present can include at least one member selected from the group consisting of polyvinyl alcohol, fish gelatin, gluten, casein, zein, and combinations thereof. The formulation can have a viscosity suitable for application and adhesion to the palm skin surface prior to evaporation of the volatile solvent system, and can form a solidified, coherent and flexible layer after at least partial evaporation of the volatile solvent system. The drug can continue to be topically delivered at the therapeutically effective rate after the volatile solvent system is at least substantially all evaporated. Other steps include leaving the formulation on the skin surface for an intended application period of at least 2 hours, and removing the solidified, coherent and flexible layer from the skin surface after the intended application period.

Additional features and advantages of the invention will be apparent from the following detailed description which illustrate, by way of example, features of the invention.

### DETAILED DESCRIPTION OF THE PREFERRED EMBODIMENTS

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a drug” includes reference to one or more of such compositions.

“Skin” is defined to include human skin (intact, diseased, ulcerous, or broken) of a subject suffering from alopecia, or hair loss. “Suffering” when referring to a patient with alopecia or hair loss includes current and likely future suffering, e.g., someone who is genetically predisposed to hair loss or alopecia, so that “skin suffering from alopecia” includes skin with alopecia in remission.

The term “drug(s)” or “drug(s) suitable for treating alopecia” refers to any bioactive agent that is applied to, into, or through the skin which is capable of stimulating hair growth. This includes compositions that are traditionally identified as drugs, as well other bioactive agents that are not always considered to be “drugs” in the classic sense. Examples of drugs which can be used in the present invention include corticosteroids such as betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, and combinations thereof. Other drugs can also be used. Other examples include drugs which can irritate the skin to stimulate hair growth such as minoxidil, spironolactone, finasteride, anthralin, tretinoin topical immunotherapeutic agents such as dinitrochlorobenzene, squaric acid dibutyl ester, diphenylcyclopropenone, other hair growth stimulants, or combinations thereof.

When referring generally to a “drug,” it is understood that there are various forms of a given drug, and those various forms are expressly included. In accordance with this, various drug forms include polymorphs, salts, hydrates, solvates, and cocrystals. For some drugs, one physical form of a drug may possess better physical-chemical properties making it more amenable for getting to, into, or through the skin, and this particular form is defined as the “physical form favorable for dermal delivery.” For example the steady state flux of diclofenac sodium from flux enabling non-volatile solvents is much higher than the steady state flux of diclofenac acid from the same flux enabling non-volatile solvents. It is therefore desirable to evaluate the flux of the physical forms of a drug from non-volatile solvents to select a desirable physical form/non-volatile solvent combination.

The phrases “dermal drug delivery” or “dermal delivery of drug(s)” shall include both transdermal and topical drug delivery, and includes the delivery of drug(s) to, through, or into the skin. “Transdermal delivery” of drug can be targeted to skin tissues just under the skin, regional tissues or organs under the skin, systemic circulation, and/or the central nervous system.

The term “flux” such as in the context of “dermal flux” or “transdermal flux,” respectively, refers to the quantity of the drug permeated into or across skin per unit area per unit time. A typical unit of flux is microgram per square centimeter per hour. One way to measure flux is to place the formulation on a known skin area of a human volunteer and measure how much drug can permeate into or across skin within certain time constraints. Various methods (in vivo methods) might be used for the measurements as well. The method described in Example 1 or other similar method (in vitro methods) can also be used to measure flux. Although an in vitro method uses human epidermal membrane obtained from a cadaver, or freshly separated skin tissue from hairless mice rather than measure drug flux across the skin using human volunteers, it is generally accepted by those skilled in the art that results from a properly designed and executed in vitro test can be used to estimate or predict the results of an in vivo test with reasonable reliability. Therefore, “flux” values referenced herein can mean that measured by either in vivo or in vitro methods.

The term “flux-enabling” with respect to the non-volatile solvent system (or solidified layer including the same) refers to a non-volatile solvent system (including one or more non-volatile solvents) selected or formulated specifically to be able to provide therapeutically effective flux for a particular drug(s). For topically or regionally delivered drugs, a flux enabling non-volatile solvent system is defined as a non-volatile solvent system which, alone without the help of any other ingredients, is capable of delivering therapeutic sufficient levels of the drug across, onto or into the subject's skin when the non-volatile solvent system is saturated with the drug. For systemically targeted drugs, a flux enabling non-volatile solvent system is a non-volatile solvent system that can provide therapeutically sufficient daily doses over 24 hours when the non-volatile solvent system is saturated with the drug and is in full contact with the subject's skin with no more than 500 cm² contact area. Preferably, the contact area for the non-volatile solvent system is no more than 100 cm². Testing using this saturated drug-in-solvent state can be used to measure the maximum flux-generating ability of a non-volatile solvent system. To determine flux, the drug solvent mixture needs to be kept on the skin for a clinically sufficient amount of time. In reality, it may be difficult to keep a liquid solvent on the skin of a human volunteer for an extended period of time. Therefore, an alternative method to determine whether a solvent system is “flux-enabling” is to measure the in vitro drug permeation across the hairless mouse skin or human cadaver skin using the apparatus and method described in Example 1. This and similar methods are commonly used by those skilled in the art to evaluate permeability and feasibility of formulations. Alternatively, whether a non-volatile solvent system is flux-enabling can be tested on the skin of a live human subject with means to maintain the non-volatile solvent system with saturated drug on the skin, and such means may not be practical for a product. For example, the non-volatile solvent system with saturated drug can be soaked into an absorbent fabric material which is then applied on the skin and covered with a protective membrane. Such a system is not practical as a pharmaceutical product, but is appropriate for testing whether a non-volatile solvent system has the intrinsic ability to provide sufficient drug flux, or whether it is flux-enabling.

It is also noted that once the formulation forms a solidified layer, the solidified layer can also be “flux enabling” for the drug while some of the non-volatile solvents remain in the solidified layer, even after the volatile solvents (including water) have been substantially evaporated.

The phrase “effective amount,” “therapeutically effective amount,” or “therapeutically effective rate(s)” of a drug refers to sufficient amounts or delivery rates of a drug which achieves appreciable level of hair growth. It is understood that “appreciable level” may or may not meet any government agencies' efficacy standards for approving the commercialization of a product. It is understood that various biological factors may affect the ability of a substance to perform its intended task. Therefore, an “effective amount,” “therapeutically effective amount,” or “therapeutically effective rate(s)” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine. For example, the therapeutically effective dermal flux of clobetasol needed for treating alopecia is estimated to be in the about 0.05 mcg/cm²/hour. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rate is well within the ordinary skill in the art of pharmaceutical sciences and medicine.

“Therapeutically effective flux” or “therapeutically sufficient flux” is defined as the permeation flux of the selected drug that delivers sufficient amount of drug into or across the skin to be clinically beneficial. “Clinically beneficial” or “clinically sufficient” when referring to flux means at some of the patient population can obtain some degree of benefit from the drug flux. It does not necessarily mean that most of the patient population can obtain some degree of benefit or the benefit is high enough to be deemed “effective” by relevant government agencies or the medical profession. More specifically, for drugs that target skin tissues close to the skin surface), “therapeutically effective flux” refers to the drug flux that can deliver a sufficient amount of the drug into the skin or tissues within a clinically reasonable amount of time. Clinically reasonable skin contact area can be defined as a size of skin application area that most subjects would accept. Typically, a skin contact area of 400 cm² or less is considered reasonable, unless there is some side effect associated with large areas of coverage. For example, it may be desirable to limit corticosteroid dermal delivery to 100 cm² to avoid unwanted transdermal side effects.

The term “plasticizing” in relation to flux-enabling non-volatile solvent(s) is defined as a flux-enabling non-volatile solvent that acts as a plasticizer for the solidifying agent. A “plasticizer” is an agent which is capable of increasing the percentage elongation of the formulation after the volatile solvent system has at least substantially evaporated. Plasticizers also have the capability to reduce the brittleness of solidified formulation by making it more flexible and/or elastic. For example, propylene glycol is a “flux-enabling, plasticizing non-volatile solvent” for the drug ketoprofen with polyvinyl alcohol as the selected solidifying agent. However, propylene glycol in a formulation of ketoprofen with Gantrez S-97 or Avalure UR 405 as solidifying agents does not provide the same plasticizing effect. The combination of propylene glycol and Gantrez S-97 or Avalure UR 405 is less compatible and results in less desirable formulation for topical applications. Therefore, whether a given non-volatile solvent is “plasticizing” depends on which solidifying agent(s) is selected.

The term “adhesion” when referring to a solidified layer herein refers to sufficient adhesion between the solidified layer and the skin so that the layer does not fall off the skin during intended use on most subjects. “Adhesive” when used to describe the solidified layer means the solidified layer is adhesive to the body surface to which the initial formulation layer was originally applied (before the evaporation of the volatile solvent(s)). In one embodiment, it does not mean the solidified layer is adhesive on the opposing side. In addition, it should be noted that whether a solidified layer can adhere to a skin surface for the desired extended period of time partially depends on the condition of the body surface. For example, excessively sweating or oily skin, or oily substances on the skin surface may make the solidified layer less adhesive to the skin. Therefore, the adhesive solidified layer of the current invention may not be able to maintain perfect contact with the body surface and deliver the drug over a sustained period of time for every subject under any conditions on the body surface. A standard is that it maintains good contact with most of the body surface, e.g. 70% of the total area, over the specified period of time for most subjects under normal conditions of the body surface and external environment.

The terms “flexible,” “elastic,” “elasticity,” or the like, as used herein refer to sufficient elasticity of the solidified layer so that it is not broken if it is stretched in at least one direction by up to about 5%, and often to about 10% or even greater. For example, a solidified layer that exhibits acceptably elasticity and adhesion to skin can be attached to human skin over a flexible skin location, e.g., elbow, finger, wrist, neck, lower back, lips, knee, etc., and will remain substantially intact on the skin upon stretching of the skin. It should be noted that the solidified layers of the present invention do not necessarily have to have any elasticity in some embodiments.

The term “peelable,” when used to describe the solidified layer, means the solidified layer can be lifted from the skin surface in one large piece or several large pieces, as opposed to many small pieces or crumbs.

The term “sustained” relates to therapeutically effective rates of dermal drug delivery for a continuous period of time of at least 30 minutes, and in some embodiments, periods of time of at least about 2 hours, 4 hours, 8 hours, 12 hours, 24 hours, or longer.

The use of the term “substantially” when referring to the evaporation of the volatile solvents means that a majority of the volatile solvents which were included in the initial formulation have evaporated. Similarly, when a solidified layer is said to be “substantially devoid” of volatile solvents, including water, the solidified layer has less than 10 wt %, and preferably less than 5 wt %, of the volatile solvents in the solidified layer as a whole.

“Volatile solvent system” can be a single solvent or a mixture of solvents that are volatile, including water and solvents that are more volatile than water. Non-limiting examples of volatile solvents that can be used in the present invention include iso-amyl acetate, denatured alcohol, methanol, ethanol, isopropyl alcohol, water, propanol, C4-C6 hydrocarbons, butane, isobutene, pentane, hexane, acetone, chlorobutanol, ethyl acetate, fluro-chloro-hydrocarbons, turpentine, methyl ethyl ketone, methyl ether, hydrofluorocarbons, ethyl ether, 1,1,1,2 tetrafluorethane 1,1,1,2,3,3,3-heptafluoropropane, 1,1,1,3,3,3 hexafluoropropane, or combinations thereof.

“Non-volatile solvent system” can be a single solvent or mixture of solvents that are less volatile than water. It can also contain substances that are solid or liquid at room temperatures, such as pH or ion-pairing agents. After evaporation of the volatile solvent system, most of the non-volatile solvent system should remain in the solidified layer for an amount of time sufficient to dermally delivery a given drug to, into, or through the skin of a subject at a sufficient flux for a period of time to provide a therapeutic effect. In some embodiments, in order to obtain desired permeability for an active drug and/or compatibility with solidifying agents or other ingredients of the formulation, a mixture of two or more non-volatile solvents can be used to form the non-volatile solvent system. In one embodiment, the combination of two or more non-volatile solvents to form a solvent system provides a higher transdermal flux for a drug than the flux provided for the drug by each of the non-volatile solvents individually. The non-volatile solvent system may also serve as a plasticizer of the solidified layer, so that the solidified layer is elastic and flexible.

The term “solvent vehicle” describes compositions that include both a volatile solvent system and non-volatile solvent system. The volatile solvent system is chosen so as to evaporate from the adhesive formulation quickly to form a solidified layer, and the non-volatile solvent system is formulated or chosen to substantially remain as part of the solidified layer after volatile solvent system evaporation so as to provide continued delivery of the drug. Typically, the drug can be partially or completely dissolved in the solvent vehicle or formulation as a whole. Likewise, the drug can also be partially or completely solubilizable in the non-volatile solvent system once the volatile solvent system is evaporated. Formulations in which the drug is only partially dissolved in the non-volatile solvent system after the evaporation of the volatile solvent system have the potential to maintain longer duration of sustained delivery, as the undissolved drug can dissolve into the non-volatile solvent system as the dissolved drug is being depleted from the solidified layer during drug delivery.

The term “adhesive” in relation to the solidified layer means it is adhesive to the skin on which the original formulation was applied, and not necessarily, and preferably not, adhesive on the other side to other objects.

“Adhesive solidifying formulation” or “solidifying formulation” refers to a composition that has a viscosity suitable for application to a skin surface prior to evaporation of its volatile solvent(s), and which can become a solidified layer after evaporation of at least a portion of the volatile solvent(s). The solidified layer, once formed, can be very durable. In one embodiment, once solidified on a skin surface, the formulation can form a peel. The peel can be a soft, coherent solid that can be removed by peeling large pieces from the skin relative to the size of the applied formulation, and often, can be peeled from the skin as a single piece. The application viscosity is typically more viscous than a water-like liquid, but less viscous than a soft solid. Examples of preferred viscosities include materials that have consistencies similar to pastes, gels, ointments, and the like, e.g., viscous liquids that flow but are not subject to spilling. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can be from about 100 cP to about 3,000,000 cP (centipoises), and more preferably from about 1,000 cP to about 1,000,000 cP.

In some embodiments of the present invention it may be desirable to add an additional agent or substance to the formulation so as to provide enhanced or increased adhesive characteristics. The additional adhesive agent or substance can be an additional non-volatile solvent or an additional solidifying agent. Non-limiting examples of substances which might be used as additional adhesion enhancing agents include copolymers of methylvinyl ether and maleic anhydride (Gantrez polymers), polyethylene glycol and polyvinyl pyrrolidone, gelatin, low molecular weight polyisobutylene rubber, copolymer of acrylsan alkyl/octylacrylamido (Dermacryl 79), and various aliphatic resins and aromatic resins.

The terms “washable” or “removed by washing” when used with respect to the adhesive formulations of the present invention refers to the ability of the adhesive formulation to be removed by the application of a washing solvent using a normal or medium amount of washing force. The required force to remove the formulations by washing should not cause significant skin irritation or abrasion. Generally, gentle washing force accompanied by the application of an appropriate washing solvent is sufficient to remove the adhesive formulations disclosed herein. The solvents which can be used for removing by washing the formulations of the present invention are numerous, but preferably are chosen from commonly acceptable solvents including the volatile solvents listed herein. Preferred washing solvents do not significantly irritate human skin and are generally available to the average subject. Examples of washing solvents include but are not limited to water, ethanol, methanol, isopropyl alcohol, acetone, ethyl acetate, propanol, or combinations thereof. In aspect of the invention the washing solvents can be selected from the group consisting of water, ethanol, isopropyl alcohol or combinations thereof. Surfactants can also be used in some embodiments.

The term “drying time” or “acceptable length of time” refer to the time it takes for the formulation to form a non-messy solidified surface after application on skin under standard skin and ambient conditions, and with standard testing procedure. It is noted that the word “drying time” in this application does not mean the time it takes to completely evaporate off the volatile solvent(s). Instead, it means the time it takes to form the non-messy solidified surface as described above.

“Standard skin” is defined as dry, healthy human skin with a surface temperature of between about 30° C. to about 36° C. Standard ambient conditions are defined by the temperature range of from 20° C. to 25° C. and a relative humidity range of from 20% to 80%. The term “standard skin” in no way limits the types of skin or skin conditions on which the formulations of the present invention can be used. The formulations of the present invention can be used to treat all types of “skin,” including undamaged (standard skin), diseased skin, or damaged skin. Although skin conditions having different characteristics can be treated using the formulations of the present invention, the use of the term “standard skin” is used merely as a standard to test the compositions of the varying embodiments of the present invention. As a practical matter, formulations that perform well (e.g., solidify, provide therapeutically effective flux, etc.) on standard skin can also perform well diseased or damaged skin.

The “standard testing procedure” or “standard testing condition” is as follows: To standard skin at standard ambient conditions is applied an approximately 0.1 mm layer of the adhesive solidifying formulation and the drying time is measured. The drying time is defined as the time it takes for the formulation to form a non-messy surface such that the formulation does not lose mass by adhesion to a piece of 100% cotton cloth pressed onto the formulation surface with a pressure of between about 5 and about 10 g/cm² for 5 seconds.

“Solidified layer” describes the solidified or dried layer of an adhesive solidifying formulation after at least a portion of the volatile solvent system has evaporated. The solidified layer remains adhered to the skin, and is preferably capable of maintaining good contact with the subject's skin for substantially the entire duration of application under standard skin and ambient conditions. The solidified layer also preferably exhibits sufficient tensile strength so that it can be peeled off the skin at the end of the application in one piece or several large pieces (as opposed to a layer with weak tensile strength that breaks into many small pieces or crumbles when removed from the skin).

As used herein, a plurality of drugs, compounds, and/or solvents may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With these definitions in mind, the present invention is drawn to an adhesive solidifying formulation for dermal delivery of a drug can comprise a drug suitable for treating alopecia, a solvent vehicle, and a solidifying agent. The solvent vehicle can comprise a volatile solvent system having one or more volatile solvent(s) and a non-volatile solvent system having one or more non-volatile solvent(s), wherein the non-volatile solvent system is preferably capable of delivering the drug at therapeutically effective rates. The formulation can have viscosity suitable for application to the skin surface as a layer prior to evaporation of at least one volatile solvent, and can further be formulated such that when applied to the skin surface, the formulation forms a solidified layer after at least a portion of the volatile solvent(s) is (are) evaporated. The drug for treating alopecia can continue to be delivered topically from the solidified layer for a sustained period of time.

In an alternative embodiment, a method of treating alopecia comprises applying an adhesive solidifying formulation to a skin surface suffering from alopecia, topically delivering the drug from the solidified layer over a sustained period of time and at desired rates, and removing the solidified layer from the skin surface after a period of time has elapsed or the desired quantity of the drug has been delivered. The formulation can include a drug, a solvent vehicle, and a solidifying agent. The solvent vehicle can comprise a volatile solvent system having one or more volatile solvent and a non-volatile solvent system having one or more non-volatile solvent, wherein the non-volatile solvent system is preferably flux-enabling. The formulation can have a viscosity suitable for application to the skin surface as a layer prior to evaporation of the volatile solvent. When the formulation is applied to the skin surface, the formulation can form a solidified layer after at least a portion of the volatile solvent system evaporated. In one embodiment, if the formulation contains sufficient amount of volatile solvent(s) whose boiling point is below 20° C., and the formulation is stored in a pressurized container, the formulation may be applied to the skin by spraying. Volatile solvent(s) whose boiling point is below 20° C. include without limitation: dimethyl ether, diethyl ether, butane, propane, 1,1, difluoroethane, 1,1,1,2 tetrafluorethane, 1,1,1,2,3,3,3-heptafluoropropane, 1,1,1,3,3,3 hexafluoropropane, and other hydrofluorocarbons. Volatile solvents whose boiling points are below 20° C. can be used as propellants in pharmaceutical and cosmetic products, and they are referred as gaseous volatile solvents herein.

In still another embodiment, a solidified layer for treating alopecia can comprise a drug for treating alopecia, a non-volatile solvent system, and a solidifying agent. The non-volatile solvent system can be flux-enabling. The solidified layer itself can also be flux-enabling (meaning the drug can be topically delivered to the skin at therapeutically rates from the solidified layer). Further, the solidified layer can have sufficient flexibility and adhesion to the skin surface so that it can maintain good contact with the skin surface to which it was originally applied for at least most of the intended duration of the application.

In another embodiment, a formulation for treating a subject suffering from alopecia can comprise a drug, a solvent vehicle, and a solidifying agent. The drug can include a member selected from the group consisting of clobetasol propionate, clobetasol, derivatives thereof, and combinations thereof. The solvent vehicle can comprise a volatile solvent system including at least one volatile solvent, and a non-volatile solvent system comprising at least one solvent selected from the group consisting of propylene glycol, glycerol, and combinations thereof, and at least one solvent selected from the group consisting of isostearic acid, oleic acid, and combinations thereof. The solidifying agent can include a member selected from the group consisting of polyvinyl alcohol, fish gelatin, gluten, casein, zein, and combinations thereof. The formulation can have a viscosity suitable for application and adhesion to a skin surface prior to evaporation of the volatile solvent system, and, after being applied to a skin surface as a layer, can form a solidified, coherent, flexible, and continuous layer after at least partial evaporation of the volatile solvent system. The continues to be topically delivered at the therapeutically effective rate after the volatile solvent system is at least substantially all evaporated.

In another embodiment, a method for treating alopecia can comprise applying to a skin area of a subject suffering from alopecia a 0.01 mm to 2 mm thick layer of an adhesive solidifying formulation. The formulation can comprise a drug including at least one member selected from the group consisting of clobetasol propionate, clobetasol, and combinations thereof, a volatile solvent system including at least one volatile solvent. Other ingredients can comprise a non-volatile solvent system including at least one solvent selected from the group consisting of propylene glycol, glycerol, and combinations thereof, and at least one solvent selected from the group consisting of isostearic acid, oleic acid, and combinations thereof. A solidifying agent can also be present can include at least one member selected from the group consisting of polyvinyl alcohol, fish gelatin, gluten, casein, zein, and combinations thereof. The formulation can have a viscosity suitable for application and adhesion to the palm skin surface prior to evaporation of the volatile solvent system, and can form a solidified, coherent and flexible layer after at least partial evaporation of the volatile solvent system. The drug can continue to be topically delivered at the therapeutically effective rate after the volatile solvent system is at least substantially all evaporated. Other steps include leaving the formulation on the skin surface for an intended application period of at least 2 hours, and removing the solidified, coherent and flexible layer from the skin surface after the intended application period.

Thus, these embodiments exemplify the present invention which is related to novel formulations, methods, and solidified layers that typically involve formulations in the initial form of semi-solids (including creams, gels, pastes, ointments, and other viscous liquids), which can be easily applied onto the skin as a layer, and can quickly (from 15 seconds to about 4 minutes under standard skin and ambient conditions) to moderately quickly (from about 4 to about 15 minutes under standard skin and ambient conditions) change into a solidified layer, e.g., a coherent and soft solid layer, for dermal drug delivery for treating alopecia. A solidified layer, which can also optionally be a peel or removable by washing, thus formed is capable of dermally delivering drug over an sustained period of time, e.g., hours to tens of hours, so that most of the drug delivery takes place after the solidified layer is formed.

Additionally, the solidified layer typically adheres to the skin, but has a solidified, minimally-adhering, outer surface which is formed relatively soon after application and which does not substantially transfer to or otherwise soil clothing or other objects or that the solidified layer may contact. The solidified layer can also be formulated such that it has sufficient flexibility and adhesion to the skin surface so that it can maintain good contact with the skin area to which it was originally applied for a sustained period of time. In selecting the various components that can be used, e.g., drug, solvent vehicle of volatile solvent system and non-volatile solvent system, solidifying agent(s), etc., various considerations can occur. For example, the volatile solvent system can be selected from pharmaceutically or cosmetically acceptable solvents known in the art. In one embodiment of the present invention, the volatile solvent system can include ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone, or combinations thereof. In another embodiment of the present invention, the volatile solvent system can include iso-amyl acetate, denatured alcohol, methanol, propanol, isobutene, pentane, hexane, chlorobutanol, turpentine, methyl ethyl ketone, or combinations thereof. The volatile solvent system can include a mixture or combination of any of the volatile solvents set forth in the embodiments above. Additionally, these volatile solvents should be chosen to be compatible with the rest of the formulation. It is desirable to use an appropriate weight percentage of the volatile solvent(s) in the formulation. Too much of the volatile solvent system prolongs the drying time. Too little of the volatile solvent system can make it difficult to spread the formulation on the skin. For most formulations, the weight percentage of the volatile solvent(s) can be from about 10 wt % to about 85 wt %, and more preferably from about 20 wt % to about 50 wt %.

The volatile solvent system can also be chosen to be compatible with the non-volatile solvent, solidifying agent, drug, and any other excipients that may be present. For example, polyvinyl alcohol (PVA) is not soluble in ethanol. Therefore, a volatile solvent which will dissolve PVA needs to be formulated in the solidifying formulation. For instance, water will dissolve PVA and can be utilized as a volatile solvent in a formulation; however, the drying time in such a formulation may be too long to certain applications. Therefore, a second volatile solvent (e.g., ethanol) can be formulated into the solidified layer to reduce the water content but maintain a sufficient amount of water to keep PVA in solution and thereby reduce the drying time for the solidifying formulation.

The non-volatile solvent system can also be chosen or formulated to be compatible with the solidifying agent, the drug, the volatile solvent, and any other ingredients that may be present. For example, the solidifying agent can be chosen so that it is dispersible or soluble in the non-volatile solvent system. Most non-volatile solvent systems and solvent vehicles as a whole will be formulated appropriately after experimentation. For instance, certain drugs have good solubility in poly ethylene glycol (PEG) having a molecular weight of 400 (PEG 400, non-volatile solvent) but poor solubility in glycerol (non-volatile solvent) and water (volatile solvent). However, PEG 400 cannot effectively dissolve poly vinyl alcohol (PVA), and thus, is not very compatible alone with PVA, a solidifying agent. In order to dissolve sufficient amount of an active drug and use PVA as a solidifying agent at the same time, a non-solvent system including PEG 400 and glycerol (compatible with PVA) in an appropriate ratio can be formulated, achieving a compatibility compromise. As a further example of compatibility, non-volatile solvent/solidifying agent incompatibility is observed when Span 20 is formulated into a formulation containing PVA. With this combination, Span 20 can separate out of the formulation and form an oily layer on the surface of the solidified layer. Thus, appropriate solidifying agent/non-volatile solvent selections are desirable in developing a viable formulation and compatible combinations.

In further detail, non-volatile solvent(s) that can be used alone or in combination to form non-volatile solvent systems can be selected from a variety of pharmaceutically acceptable liquids. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, or combinations thereof. In one embodiment of the present invention, the non-volatile solvent system can include glycerol, propylene glycol, isostearic acid, oleic acid, propylene glycol, trolamine, tromethamine, triacetin, sorbitan monolaurate, sorbitan monooleate, sorbitan monopalmitate, butanol, or combinations thereof. In another embodiment, the non-volatile solvent system can include benzoic acid, butyl alcohol, dibutyl sebecate, diglycerides, dipropylene glycol, eugenol, fatty acids such as coconut oil, fish oil, palm oil, grape seed oil, isopropyl myristate, mineral oil, oleyl alcohol, vitamin E, triglycerides, sorbitan fatty acid surfactants, triethyl citrate, or combinations thereof. In a further embodiment the non-volatile solvent system can include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG Fatty acid esters such as PEG-stearate, PEG-oleate, PEG-laurate, PEG fatty acid diesters such as PEG-dioleate, PEG-distearate, PEG-castor oil, glyceryl behenate, PEG glycerol fatty acid esters such as PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG -alkyl ethers such as PEG-cetyl ether, PEG-stearyl ether, PEG-sorbitan fatty acid esters such as PEG-sorbitan diisosterate, PEG-sorbitan monostearate, propylene glycol fatty acid esters such as propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, or combinations thereof. In yet a further embodiment, the non-volatile solvent system can include a combination or mixture of non-volatile solvents set forth in the any of the above discussed embodiments.

In addition to these and other considerations, the non-volatile solvent system can also serve as plasticizer in the adhesive formulation so that when the solidified layer is formed, the layer is soft, flexible, stretchable, and/or otherwise “skin friendly.”

In one embodiment, the non-volatile solvent system can comprise propylene glycol. In another embodiment the non-volatile solvent system can comprise isostearic acid. In another embodiment, the non-volatile solvent system can comprise propylene glycol and isostearic acid. In yet another embodiment the non-volatile solvent system comprises propylene glycol and isostearic acid in a weight ratio of from 19:1 to 4:1.

Certain volatile and/or nonvolatile solvent(s) that are irritating to the skin may be desirable to use to achieve the desired solubility and/or permeability of the drug. It is also desirable to add compounds that are both capable of preventing or reducing skin irritation and are compatible with the formulation. For example, in a formulation where the volatile solvent is capable of irritating the skin, it would be helpful to use a non-volatile solvent that is capable of reducing skin irritation. Examples of solvents that are known to be capable of preventing or reducing skin irritation include, but are not limited to, glycerin, honey, and propylene glycol.

The formulations of the current invention may also contain two or more non-volatile solvents that independently are not enabling non-volatile solvents for a drug but when formulated together become an enabling non-volatile solvent. One possible reason for these initially non enabling non-volatile solvents to become enabling non-volatile solvents when formulated together may be due to the optimization of the ionization state of the drug to a physical form which has higher flux or the non-volatile solvents act in some other synergistic manner. One further benefit of the mixing of the non-volatile solvents is that it may optimize the pH of the formulation or the skin tissues under the formulation layer to minimize irritation. Examples of suitable combinations of non-volatile solvents that result in an adequate non-volatile solvent system include but are not limited to isostearic acid/trolamine, isostearic acid/diisopropyl amine, oleic acid/trolamine, and propylene glycol/isostearic acid.

The selection of the solidifying agent can also be carried out in consideration of the other components present in the adhesive formulation. The solidifying agent can be selected or formulated to be compatible to the drug and the solvent vehicle (including the volatile solvent(s) and the non-volatile solvent system), as well as to provide desired physical properties to the solidified layer once it is formed. Depending on the drug, solvent vehicle, and/or other components that may be present, the solidifying agent can be selected from a variety of agents. In one embodiment, the solidifying agent can include polyvinyl alcohol with a MW range 20,000-70,000 (Amresco), esters of polyvinylmethylether/maleic anhydride copolymer (ISP Gantrez ES-425 and Gantrez ES-225) with MW 80,000-160,000, neutral copolymer of butyl methacrylate and methyl methacrylate (degussa Plastoid B) with MW range of 120,000-180,000, dimethylaminoethyl methacrylate-butyl methacrylate-methyl methacrylate copolymer (degussa Eudragit E100) MW 100,000-200,000, ethyl acrylate-methyl methacrylate-trimethylammonioethyl methacrylate chloride copolymer MW greater than 5,000 or similar MW to Eudragit RLPO (degussa), Zein (prolamine), MW greater than 5,000 (zein, MW around 35,000, Freeman industries), pregelatinized starch having a MW similar to Instant Pure-Cote B793 (Grain Processing Corporation), ethyl cellulose MW greater than 5,000 or MW similar to Aqualon EC N7, N10, N14, N22, N50, or N100 (Hercules), fish gelatin having a MW 20,000-250,000 (Norland Products), gelatin, other animal sources with MW greater than 5,000, acrylates/octylacrylamide copolymer MW greater than 5,000 or MW similar to National Starch and Chemical Dermacryl 79.

In another embodiment the solidifying agent can include ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, or combinations thereof. In a further embodiment, the solidifying agent can include ammonia methacrylate, carrageenan, cellulose acetate phthalate aqueous such as CAPNF from Eastman, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers such as BASF's Kollicoat polymers, methacrylic acid and methacrylate based polymers such as poly(methacrylic acid), or combinations thereof. In yet a further embodiment the solidifying agent can include a combination of solidifying agents set forth in the any of the above discussed embodiments. Other polymers may also be suitable as the solidifying agent, depending on the solvent vehicle components, the drug, and the specific functional requirements of the given formulation. Other solidifying agents may also be suitable as the solidifying agent, depending on the solvent vehicle components, the drug, and the specific functional requirements of the given formulation.

In one embodiment, the non-volatile solvent system and the solidifying agent(s) should be compatible with each other. Compatibility can be defined as i) the solidifying agent does not substantially negatively influence the function of the non-volatile solvent system, except for some reduction of flux; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness. The weight ratio of the non-volatile solvent system to the solidifying agent(s) can be from about 0.1:1 to about 10:1. In another aspect, the ratio between the non-volatile solvent system and the solidifying agent can be from about 0.5:1 to about 2:1.

The thickness of the formulation layer applied on the skin should also be appropriate for a given formulation and desired drug delivery considerations. If the layer is too thin, the amount of the drug may not be sufficient to support sustained delivery over the desired length of time. If the layer is too thick, it may take too long to form a non-messy outer surface of the solidified layer. If the drug is very potent and the solidified layer has very high tensile strength, a layer as thin as 0.01 mm may be sufficient. If the drug has rather low potency and the solidified layer has low tensile strength, a layer as thick as 2-3 mm may be desirable. Thus, for most drugs and formulations, the appropriate thickness can be from about 0.05 mm to about 3 mm, 0.1 mm to about 2 mm, or from about 0.2 mm to about 0.4 mm. In one embodiment, the formulations of the present invention can have sufficient gas volatile solvents such that the formulation can be contained in a pressurized container and applied to the skin by spraying. In another embodiment, the formulation can be sprayed on a skin surface of a subject experiencing alopecia.

The formulations of the present invention can be applied over a variety of skin surfaces of subjects suffering from alopecia. Generally, the skin surface can be any size; however, in one embodiment, it can be desirable to limit the area to no more than 100 cm², and often, no more than 20 cm², particularly if the active drug is a corticosteroid. The desirability of limiting the skin area is based on fact that though the corticosteroid is delivered topically, a good portion of the delivered drug may enter the systemic circulation which, if at high enough quantities, can cause undesirable side effects. Therefore, it can be desirable to achieve a balance of good topical effect and minimum systemic drug absorption. Since the acting site of the corticosteroids is the hair follicles which exist somewhat deep under the skin surface, the drug needs to travel along a relatively long path through the skin, and thus, the subject can experience some systemic uptake. This being stated, a significant number of alopecia subjects only have relatively small skin area where they are experiencing alopecia. Therefore, a limitation on the skin treatment area can stratify the treatment need of those subjects, while minimizing systemic side effects. Even for subjects who alopecia skin areas are larger, a limitation on treatment skin area may still make sense as it allows the alopecia skin areas to be treated portion by portion with minimized potential of systemic side effects. Treating large alopecia skin areas portion by portion is possible because the treatment of each portion is expected to be periodical instead of continuous.

A further feature of a formulation prepared in accordance with embodiments of the present invention is related to drying time. If a formulation dries too quickly, the user may not have sufficient time to spread the formulation into a thin layer on the skin surface before the formulation is solidified, leading to poor skin contact. If the formulation dries too slowly, the subject may have to wait a long time before resuming normal activities (e.g. putting clothing on) that may remove un-solidified formulation. Thus, it is desirable for the drying time to be longer than about 15 seconds but shorter than about 15 minutes. In one embodiment, the drying time for the formulation is less than about 10 minutes. In another embodiment the drying time is from 0.5 minutes to about 5 minutes.

Another feature of the formulations of the present invention is related to solidifying formulations comprising a drug for treating alopecia, a non-volatile solvent system comprising at least one non-volatile solvent, a solidifying agent, and a volatile solvent system comprising a volatile solvent whose boiling point is below 20° C. (such a solvent is referred to as gaseous volatile solvent hereafter). The formulation can be stored in a pressurized container and be sprayed on the skin surface with the help of the gaseous volatile solvent. Some hydrofluorocarbons used as gaseous volatile solvents in pharmaceutical or cosmetic industries can work in this design. More specifically, the gaseous volatile solvents may include, but not limited to dimethyl ether, butane, 1,1, difluoroethane, 1,1,1,2 tetrafluorethane, 1,1,1,2,3,3,3-heptafluoropropane, 1,1,1,3,3,3 hexafluoropropane, or combinations thereof. The formulation may also be expelled out of the container and applied on the skin via a manual pump. Formulations including a gaseous volatile solvent are expected to “dry” much faster. Spraying the formulation onto the skin suffering from alopecia can be more convenient when applying to an area with some hair loss.

The formulations of the current invention may further comprise a pH modifying agent for adjusting the pH of the formulation to a point or a range most suitable for the delivery of the drug. This feature can be valuable for a drug that is ionizable.

As a further note, it is a unique feature of the solidified layers of the present invention that they can keep a substantial amount of the non-volatile solvent system, which is optimized for delivering the drug, on the body surface. This feature can provide unique advantages over existing products. For example, in some semi-solid formulations, upon application to a skin surface the volatile solvents quickly evaporate and the formulation layer solidifies into a hard lacquer-like layer. The drug molecules are immobilized in the hard lacquer layer and are substantially unavailable for delivery into the skin surface. As a result, it is believed that the delivery of the drug is not sustained over a long period of time. In contrast to this type of formulation, the solidified layers formed using the formulations of the present invention keep the drug molecules quite mobile in the non-volatile solvent system which is in contact with the skin surface, thus ensuring sustained delivery.

These and other advantages can be summarized in the following non-limiting list of benefits, as follows. The adhesive solidifying formulations of the present invention can be in an initial semisolid form that is easy to be applied on a skin surface as a layer. Additionally, upon volatile solvent system evaporation, a solidified layer is formed which is relatively thick and can contain much more active drug than a typical layer of traditional cream, gel, lotion, ointment, paste, etc., and further, is not as subject to unintentional removal. Further, as the solidified layer remains adhesive to skin surface, good contact with the skin can be maintained. The solidified layer of the present invention can also be removed by washing or by peeling. The ability to remove by washing can be particularly advantageous when treating areas which have some hair or which areas are responding to the hair stimulation treatment. The solidified layer can have sufficient flexibility and adhesion to the skin surface so that it can maintain good contact with the skin surface to which it was originally applied for a sustained period of time. Still further, the drug can be topically delivered from the solidified layer at therapeutically effective rates over a sustained period of time, and can be easily removed by washing without removing any significant amounts of hair.

Examples of hair stimulating drugs which can be used in the present invention include corticosteroids such as betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, or combinations thereof. Other drugs can also be used, including those which can irritate the skin to stimulate hair growth such as minoxidil, spironolactone, finasteride, anthralin, tretinoin, topical immunotherapeutic agents such as dinitrochlorobenzene, squaric acid dibutyl ester, diphenylcyclopropenone, other hair growth stimulants, or combinations thereof.

In one embodiment, the hair growth stimulating drug can include clobetasol or a derivative thereof. Examples of clobetsol derivatives include clobetasol propionate and clobetasol butyrate. In one embodiment, the drug can include clobetasol propionate. When the drug is clobetasol or a derivative thereof, the concentration of the drug in the formulation can be greater than 0.03 wt %. In one embodiment, the concentration of the clobetsol or derivative thereof in the formulation can be greater than 0.14 wt %. In another embodiment, the concentration of the clobetsol or derivative thereof in the formulation is greater than 0.29 wt %. The higher clobetasol concentrations can potentially deliver the drug at higher flux (higher quantities per unit time and per unit skin area), which can result in better efficacy. The high clobetasol concentration formulations are especially suitable in situations where the alopecia skin area is small; since high flux across a small skin area will not result in high systemic absorption (systemic absorption is proportional to flux times the skin area).

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Hairless mouse skin (HMS) or human epidermal membrane (HEM) is used as the model membranes as noted for the in vitro flux studies described in herein. Hairless mouse skin (HMS) is used as the model membrane for the in vitro flux studies described in herein. Freshly separated epidermis removed from the abdomen of a hairless mouse is mounted carefully between the donor and receiver chambers of a Franz diffusion cell. The receiver chamber is filled with pH 7.4 phosphate buffered saline (PBS). The experiment is initiated by placing test formulations (of Examples 2-5) on the stratum corneum (SC) of the skin sample. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time. It is to be noted that human cadaver skin can be used as the model membrane for the in vitro flux studies as well. The mounting of the skin and the sampling techniques used as the same as described above for the HMS studies.

### Example 2

Formulations of betamethasone dipropionate (BDP) in various non-volatile solvent systems are evaluated following procedure described in Example 1. Excess BDP is present. The flux of BDP from the test formulations through HEM is presented in Table 1 below.

TABLE 1
Non-volatile solvents for betamethasone dipropionate

| Non-volatile solvent system | Skin Flux* (ng/cm²/h) |
|---|---|
| Propylene Glycol | 195.3 ± 68.5 |
| Triacetin | 4.6 ± 2.8 |
| Light Mineral Oil | 11.2 ± 3.1 |
| Oleic acid | 8.8 ± 3.3 |
| Sorbitan Monolaurate | 30.0 ± 15.9 |
| Labrasol | 12.2 ± 6.0 |

*Skin flux measurements represent the mean and standard deviation of three determinations. Flux measurements reported were determined from the linear region of the cumulative amount versus time plots. The linear region was observed to be between 6-28 hours. If the experiment was continued it is anticipated the steady state would continue.

Active enzymes in the skin convert BDP to betamethasone. The steady state flux values reported in Table 1 are quantified using external betamethasone standards and are reported as amount of betamethasone permeating per unit area and time. As seen from the results triacetin, labrasol, oleic acid, and light mineral oil have flux values close to 10 ng/cm²/hr. Addition of solidifying agents and other components could possibly decrease the flux and therefore the above mentioned solvents would not be an ideal non-volatile solvent. However, sorbitan monolaurate and propylene glycol have average flux of 30 ng/cm²/hr and 195 ng/cm²/hr, respectively, and therefore are good candidates for non-volatile solvent.

### Example 3

Formulations of clobetasol propionate in various non-volatile solvent systems are evaluated. All solvents have 0.1% (w/w) clobetasol propionate. The flux of clobetasol from the test formulations through HEM is presented in Table 2 below.

TABLE 2
Non-volatile solvents for clobetasol propionate

| Non-volatile solvent system | Skin Flux* (ng/cm²/h) |
|---|---|
| Propylene Glycol | 3.8 ± 0.4 |
| Glycerol | 7.0 ± 4.1 |
| Light Mineral Oil | 31.2 ± 3.4 |
| Isostearic acid (ISA) | 19.4 ± 3.2 |
| Ethyl Oleate | 19.4 ± 1.6 |
| Olive oil | 13.6 ± 3.3 |
| Propylene Glycol/ISA (9:1) | 764.7 ± 193.9 |

*Skin flux measurements represent the mean and standard deviation of three determinations. Flux measurements reported were determined from the linear region of the cumulative amount versus time plots. The linear region was observed to be between 6-28 hours. If the experiment was continued it is anticipated the steady state would continue.

All the neat non-volatile solutions studied have an average flux of less than 40 ng/cm²/hr over the 30 hour time period. Propylene glycol and glycerol have the lowest permeation for clobetasol propionate. A mixture of propylene glycol and isostearic acid in weight ratio of 9:1 have significantly higher flux than either of the solvents alone or the other solvents tested. The average flux is 20 times higher than that with light mineral oil which is the best non-mixed solvent. Hence, for clobetasol the propionate propylene glycol/isostearic acid combination forms a good non-volatile solvent system. This is an example that a special mixture of two or more non-volatile solvents, typically determined through experimentation, can sometimes have high flux-generating power than any single component solvent in the system.

### Examples 4-9

Adhesive solidifying formulations containing 0.05% (w/w) clobetasol propionate with propylene glycol and isostearic acid as the non-volatile solvent system and various solidifying agents are prepared. The formulations are prepared from the ingredients as shown in Table 3.

TABLE 3
Solidifying formulation components

| | | | | Percent | Percent | |
|---|---|---|---|---|---|---|
| Exam- | | Percent | Percent | Propylene | Isostearic | Percent |
| ple | Polymer | Polymer | Ethanol | glycol | acid | Water |
| 4 | Polyvinyl | 20 | 30 | 19.6 | 0.4 | 30 |
| | alcohol | | | | | |
| 5 | Shellac | 50 | 30 | 19.6 | 0.4 | 0 |
| 6 | Dermacryl | 65.76 | 21.16 | 12.76 | 0.26 | 0 |
| | 79 | | | | | |
| 7 | Eudragit | 50 | 30 | 19.6 | 0.40 | 0 |
| | E100 | | | | | |
| 8 | Eudragit | 50 | 30 | 19.6 | 0.40 | 0 |
| | RLPO | | | | | |
| 9 | Gantrez | 14.3 | 57.1 | 28 | 0.6 | 0 |
| | S97 | | | | | |

Each of the compositions shown above are studied for flux of clobetasol propionate as shown in Table 4 as follows:

TABLE 4
Steady state flux of Clobetasol propionate
through human cadaver skin at 35° C.

| Formulation | Skin Flux* (ng/cm²/h) |
|---|---|
| Example 4 | 87.8 ± 21.4 |
| Example 5 | 9.7 ± 2.4 |
| Example 6 | 8.9 ± 0.8 |
| Example 7 | 3.2 ± 1.7 |
| Example 8 | 20.2 ± 18.6 |
| Example 9 | 147.5 ± 38.8 |

*Skin flux measurements represent the mean and standard deviation of three determinations. Flux measurements reported were determined from the linear region of the cumulative amount versus time plots. The linear region was observed to be between 6-28 hours. If the experiment was continued it is anticipated the steady state would continue.

As seen from Table 4 formulation described in Example 4 that contains polyvinyl alcohol as solidifying agent has high flux of clobetasol propionate.

Polyvinyl alcohol is known to form stretchable films and it is likely that this formulation will have acceptable wear properties. The toughness of the resulting film can be modified by adding appropriate plasticizers if needed. Tackiness can also be modified by adding appropriate amounts of tackifier or by adding appropriate amounts of another solidifying agent such as dermacryl 79.

Regarding formulation described in Example 9, a higher percentage of ethanol is needed to dissolve the polymer. However, the solidifying agent used in Example 9 provides the highest flux of clobetasol propionate among the solidifying agents studied. The wear properties of this formulation can be modified by adding appropriate levels of other ingredients including but not limited to plasticizers, tackifiers, non-volatile solvents and or solidifying agents.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. An formulation for treating a subject suffering from alopecia, comprising:
a) a drug for treatment of alopecia,
b) a solvent vehicle, comprising
i) a volatile solvent system comprising at least one volatile solvent, and
ii) a non-volatile solvent system comprising at least one non-volatile solvent
c) a solidifying agent,
wherein the formulation has a viscosity suitable for application and adhesion to a skin surface prior to evaporation of the volatile solvent system, and wherein the formulation applied to the skin surface as a layer forms a solidified layer after at least partial evaporation of the volatile solvent system, wherein the drug continues to be delivered after the volatile solvent system is at least substantially evaporated.

2. A formulation as in claim 1, wherein the non-volatile solvent system is capable of delivering the drug at therapeutically effective rates.

3. A formulation as in claim 1, wherein the solidified layer is capable of delivering the drug at therapeutically effective rates over a sustained period of time of at least 2 hours.

4. A formulation as in claim 1, wherein the drug includes a corticosteriod.

5. A formulation as in claim 1, wherein the drug includes a hair growth stimulant.

6. A formulation as in claim 1, wherein the drug is a corticosteroid including a member selected from the group consisting of steroids including betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, and combinations thereof.

7. A formulation as in claim 1, wherein the drug is a substance which irritates the skin to stimulate hair growth, and includes a member selected from the group consisting of minoxidil, spironolactone, finasteride, anthralin, tretinoin topical, immunotherapeutic agents such as dinitrochlorobenzene, squaric acid dibutyl ester, diphenylcyclopropenone, and combinations thereof.

8. A formulation as in claim 1, wherein the drug includes clobetasol or a clobetasol derivative.

9. A formulation as in claim 1, wherein the drug includes clobetasol propionate.

10. A formulation as in claim 4, wherein the concentration of clobetasol or clobetasol derivative is greater than about 0.03%.

11. A formulation as in claim 4, wherein the concentration of clobetasol or clobetasol derivative is greater than 0.14%.

12. A formulation as in claim 4, wherein the concentration of clobetasol or clobetasol derivative is greater than 0.29%.

13. A formulation as in claim 1, wherein the non-volatile solvent system acts as a plasticizer for the solidifying agent.

14. A formulation as in claim 1, wherein the volatile solvent system comprises water.

15. A formulation as in claim 1, wherein the volatile solvent system comprises at least one solvent selected from the group consisting of ethanol, isopropyl alcohol, and combinations thereof.

16. A formulation as in claim 1, wherein the volatile solvent system comprises at least one solvent more volatile than water, and includes a member selected from the group consisting of ethanol, isopropyl alcohol, water, dimethyl ether, diethyl ether, butane, propane, isobutene, 1,1, difluoroethane, 1,1,1,2 tetrafluorethane, 1,1,1,2,3,3,3-heptafluoropropane, 1,1,1,3,3,3 hexafluoropropane, other hydrofluorocarbons, ethyl acetate, acetone and combinations thereof.

17. A formulation as in claim 1, wherein the volatile solvent system comprises at least one solvent more volatile than water, and includes a member selected from the group consisting of iso-amyl acetate, denatured alcohol, methanol, propanol, isobutene, pentane, hexane, chlorobutanol, turpentine, methyl ethyl ketone, and combinations thereof.

18. A formulation as in claim 1, wherein the volatile solvent system comprises at least one liquid volatile solvent and at least one gas volatile solvent having a boiling point less than 20° C.

19. A formulation as in claim 1, wherein the non-volatile solvent system includes at least one solvent selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

20. A formulation as in claim 1, wherein the non-volatile solvent system includes at least one solvent selected from the group consisting of benzoic acid, butyl alcohol, dibutyl sebecate, diglycerides, dipropylene glycol, eugenol, fatty acids such, isopropyl myristate, mineral oil, oleyl alcohol, vitamin E, triglycerides, sorbitan fatty acid surfactants, triethyl citrate, and combinations thereof.

21. A formulation as in claim 1, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

22. A formulation as in claim 1, wherein the non-volatile solvent system comprises isostearic acid.

23. A formulation as in claim 1, wherein the non-volatile solvent system comprises propypene glycol.

24. A formulation as in claim 1, wherein the non-volatile solvent system comprises propylene glycol and isostearic acid.

25. A formulation as in claim 1, wherein the non-volatile solvent system comprises propylene glycol and isostearic acid in the weight ratio of from 19:1 to 4:1.

26. A formulation as in claim 1, wherein the solidifying agent includes at least member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylam monioethyl methacrylate chloride copolymers, prolamine (Zein), pregelatinized starch, ethyl cellulose, fish gelatin, gelatin, acrylates/octylacrylamide copolymers, and combinations thereof.

27. A formulation as in claim 1, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof.

28. A formulation as in claim 1, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

29. A formulation as in claim 1, wherein the drug is clobetasol or a clobetasol derivative and the solidifying agent includes a member selected from the group consisting of gelatin, zein, gluten, polyvinyl alcohol, and combinations thereof.

30. A formulation as in claim 1, wherein the formulation is formulated to deliver the drug at a therapeutically effective rate for at least about 2 hours following the formation of the solidified layer.

31. A formulation as in claim 1, wherein the formulation is formulated to deliver the drug at a therapeutically effective rate for at least about 4 hours following the formation of the solidified layer.

32. A formulation as in claim 1, wherein the formulation is formulated to deliver the drug at a therapeutically effective rate for at least about 8 hours following the formation of the solidified layer.

33. A formulation as in claim 1, wherein the formulation is formulated to deliver the drug at a therapeutically effective rate for at least about 12 hours following the formation of the solidified layer.

34. A formulation as in claim 1, wherein the weight ratio of the non-volatile solvent system to the solidifying agent is from about 0.1:1 to about 10:1.

35. A formulation as in claim 1, wherein the weight ratio of the non-volatile solvent system to the solidifying agent is from about 0.5:1 to about 2:1.

36. A formulation as in claim 1, wherein the solidified layer is formed within about 15 minutes of application to the skin surface under standard skin and ambient conditions.

37. A formulation as in claim 1, wherein the solidified layer is formed within about 10 minutes of the application to the skin surface under standard skin and ambient conditions.

38. A formulation as in claim 1, wherein the formulation has an initial viscosity prior to skin application from about 100 cP to about 3,000,000 cP.

39. A formulation as in claim 1, wherein the formulation has an initial viscosity prior to skin application from about 1,000 cP to about 200,000 cP.

40. A formulation as in claim 1, wherein the formulation has an initial viscosity prior to skin application from about 10,000 cP to about 100,000 cP.

41. A formulation as in claim 1, wherein the weight percentage of the volatile solvent system is from about 10 wt % to about 85 wt %.

42. A formulation as in claim 1, wherein the weight percentage of the volatile solvent system is from about 20 wt % to about 50 wt %.

43. A formulation as in claim 1, wherein the non-volatile solvent includes at least two non-volatile solvents, and wherein one of the at least two non-volatile solvents is included to improve compatibility with the solidifying agent.

44. A formulation as in claim 1, wherein the solidified layer is coherent, flexible, and continuous.

45. A formulation as in claim 1, wherein the solidified layer, upon formation, is a soft, coherent solid that is peelable from a skin surface as a single piece or as only a few large pieces relative to the application size.

46. A formulation as in claim 1, wherein the solidified layer is sufficiently soluble in water that it can be washed off in a normal shower.

47. A formulation as in claim 1, wherein the solidified layer is washable with water, surfactant, alcohol, or combination thereof.

48. A formulation as in claim 1, wherein the volatile solvent system comprises a volatile solvent whose boiling point is below 20° C.

49. A formulation as in claim 48, wherein the volatile solvent with the boiling point below 20° C. is completely dissolved in the formulation.

50. A formulation as in claim 48, wherein the volatile solvent with the boiling point below 20° C. is included in the formulation as a propellant for pressurized spray-on application.

51. A formulation as in claim 48, wherein the volatile solvent with the boiling point below 20° C. is a hydrofluorocarbon.

52. The formulation as in claim 48, wherein the at least one solvent whose boiling point is below 20° C. includes a solvent selected from the group consisting of dimethyl ether, propane, isobutene, butane, 1,1, difluoroethane, 1,1,1,2 tetrafluorethane, 1,1,1,2,3,3,3-heptafluoropropane, 1,1,1,3,3,3 hexafluoropropane, and combinations thereof.

53. A method for treating alopecia, comprising
a) applying a layer of a formulation to a human skin surface of a subject suffering from alopecia, the formulation comprising:
i) a drug suitable for treating alopecia,
ii) a solvent vehicle, comprising:
a volatile solvent system including at least one volatile solvent, and
a non-volatile solvent system including at least one non-volatile solvent, wherein the non-volatile solvent system is capable of facilitating dermal delivery of the drug at a therapeutically effective rate over a sustained period of time, and
iii) a solidifying agent,
wherein the formulation has a viscosity suitable for application and adhesion to the skin surface prior to evaporation of the volatile solvent system;
b) solidifying the formulation to form a solidified layer on the skin surface by at least partial evaporation of the volatile solvent system; and
c) dermally delivering the drug from the solidified layer to the skin at therapeutically effective rates over a sustained period of time.

54. A method as in claim 53, wherein the thickness of the layer of formulation applied on the skin is between about 0.05 mm to about 3 mm.

55. A method as in claim 53, wherein the thickness of the layer of formulation applied on the skin is between about 0.1 mm to about 2 mm.

56. A method as in claim 53, wherein the thickness of the layer of formulation applied on the skin is between about 0.2 mm to about 0.6 mm.

57. A method as in claim 53, wherein formulation is applied on the skin of the subject before sleeping and the solidified layer is removed after waking.

58. A method as in claim 53, wherein the human skin covered by the formulation has an area of no more than 100 cm².

59. A method as in claim 53, wherein the human skin covered by the formulation has an area of no more than 20 cm².

60. A method as in claim 53, wherein the drug is a corticosteroid including a member selected from the group consisting of betamethasone dipropionate, halobetasol propionate, diflorasone diacetate, triamcinolone acetonide, desoximethasone, fluocinonide, halcinonide, mometasone furoate, betamethasone valerate, fluocinonide, fluticasone propionate, triamcinolone acetonide, fluocinolone acetonide, flurandrenolide, desonide, hydrocortisone butyrate, hydrocortisone valerate, alclometasone dipropionate, flumethasone pivolate, hydrocortisone, hydrocortisone acetate, and combinations thereof.

61. A method as in claim 53, wherein the drug is a substance which irritates the skin to stimulate hair growth, and includes a member selected from the group consisting of minoxidil, spironolactone, finasteride, anthralin, tretinoin topical immunotherapeutic agents such as dinitrochlorobenzene, squaric acid dibutyl ester, diphenylcyclopropenone, and combinations thereof.

62. A method as in claim 53, wherein the drug includes clobetasol or a clobetasol derivative.

63. A method as in claim 53, wherein the concentration of clobetasol or derivative of clobetasol is greater than 0.03%.

64. A method as in claim 53, wherein the concentration of clobetasol or derivative of clobetasol is greater than 0.14%.

65. A method as in claim 53, wherein the concentration of clobetasol or derivative of clobetasol is greater than 0.29%.

66. A method as in claim 53, wherein the non-volatile solvent system acts as a plasticizer for the solidifying agent.

67. A method as in claim 53, wherein the volatile solvent system comprises water.

68. A method as in claim 53, wherein the volatile solvent system includes at least one member selected from the group consisting of ethanol, isopropyl alcohol, water, dimethyl ether, diethyl ether, butane, 1-propane, isobutene, 1,1, difluoroethane, 1,1,1,2 tetrafluorethane, 1,1,1,2,3,3,3-heptafluoropropane, 1,1,1,3,3,3 hexafluoropropane, ethyl acetate, acetone and combinations thereof.

69. A method as in claim 53, wherein the volatile solvent system includes at least one member selected from the group consisting of iso-amyl acetate, denatured alcohol, methanol, propanol, isobutene, pentane, hexane, chlorobutanol, turpentine, methyl ethyl ketone, and combinations thereof.

70. A method as in claim 53, wherein the volatile solvent system comprises at least one liquid volatile solvent and at least one gaseous volatile solvent having a boiling point less than 20° C.

71. A method as in claim 70, wherein the gaseous volatile solvent is selected from the group consisting of dimethyl ether, propane, isobutene, butane, 1,1, difluoroethane, 1,1,1,2 tetrafluorethane, 1,1,1,2,3,3,3-heptafluoropropane, 1,1,1,3,3,3 hexafluoropropane, and combinations thereof.

72. A method as in claim 70, wherein the application of the formulation comprises spraying the formulation on the skin.

73. A method as in claim 53, wherein the non-volatile solvent system includes at least one member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

74. A method as in claim 53, wherein the non-volatile solvent system includes at least one member selected from the group consisting of benzoic acid, butyl alcohol, dibutyl sebecate, diglycerides, dipropylene glycol, eugenol, fatty acids such, isopropyl myristate, mineral oil, oleyl alcohol, vitamin E, triglycerides, sorbitan fatty acid surfactants, triethyl citrate, and combinations thereof.

75. A method as in claim 53, wherein the non-volatile solvent system includes at least one member selected from the group consisting of 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars, ginger extract, glycerin, high fructose corn syrup, hydrogenated castor oil, IP palmitate, lemon oil, lime oil, limonene, milk, monoacetin, monoglycerides, nutmeg oil, octyidodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof.

76. A method as in claim 53, wherein the non-volatile solvent system comprises isostearic acid.

77. A method as in claim 53, wherein the non-volatile solvent system comprises propypene glycol.

78. A method as in claim 53, wherein the non-volatile solvent system comprises propylene glycol and isostearic acid.

79. A method as in claim 53, wherein the solidifying agent includes at least one member selected from the group consisting of polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylammonioethyl methacrylate chloride copolymers, prolamine, pregelatinized starch, ethyl cellulose, fish gelatin, gelatin, zein, gluten, acrylates/octylacrylamide copolymers, and combinations thereof.

80. A method as in claim 53, wherein the solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate, and combinations thereof.

81. A method as in claim 53, wherein the solidifying agent includes at least one member selected from the group consisting of ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

82. A method as in claim 53, wherein the formulation is left on the skin surface for at least about 2 hours following the formation of the solidified layer.

83. A method as in claim 53, wherein the formulation is left on the skin surface for at least about 12 hours following the formation of the solidified layer.

84. A method as in claim 53, wherein the weight ratio of the non-volatile solvent system to the solidifying agent is from about 0.5:1 to about 2:1.

85. A method as in claim 53, wherein the solidified layer is formed within about 15 minutes of application to the skin surface under standard skin and ambient conditions.

86. A method as in claim 53, wherein the formulation has an initial viscosity prior to skin application from about 1,000 cP to about 1,000,000 cP.

87. A method as in claim 53, wherein the weight percentage of the volatile solvent system is from about 10 wt % to about 85 wt %.

88. A method as in claim 53, wherein the non-volatile solvent includes at least two non-volatile solvents, and wherein one of the at least two non-volatile solvents is included to improve compatibility with the solidifying agent.

89. A method as in claim 53, wherein the solidified layer is coherent, flexible, and continuous.

90. A method as in claim 53, wherein the solidified layer, upon formation, is a soft, coherent solid that is peelable from a skin surface as a single piece or as only a few large pieces relative to the application size.

91. A method as in claim 53, wherein the solidified layer is sufficiently soluble in water that it can be washed off in a normal shower.

92. A method as in claim 53, wherein the solidified layer is washable with water, surfactant, alcohol, or combination thereof.

93. A solidified layer for treating a subject suffering from alopecia, comprising
a) a drug for treating alopecia,
b) a non-volatile solvent system comprising at least one non-volatile solvent, wherein the non-volatile solvent system is capable of facilitating the delivery of the drug at therapeutically effective rates over a sustained period of time; and
c) a solidifying agent.

94. A solidified layer as in claim 93, wherein at least one of the non-volatile solvents in the non-volatile solvent system acts as a plasticizer for the solidifying agent.

95. A solidified layer as in claim 93, wherein solidified layer is sufficiently adhesive and flexible to remain substantially intact on a skin surface.

96. A solidified layer as in claim 93, wherein the weight ratio of the non-volatile solvent system to the solidifying agent is from about 0.5:1 to about 2:1.

97. A solidified layer as in claim 93, wherein the solidified layer is formed within 15 minutes of the application to the skin surface under standard skin and ambient conditions.

98. A solidified layer as in claim 93, wherein the solidified layer has a thickness from about 0.01 mm to about 1 mm.

99. A solidified layer as in claim 93, wherein the non-volatile solvent system includes a member selected from the group consisting of glycerol, propylene glycol, isostearic acid, oleic acid, propylene glycol, trolamine, tromethamine, triacetin, sorbitan monolaurate, sorbitan monooleate, sorbitan monopalmitate, butanol, and combinations thereof.

100. A solidified layer as in claim 93, wherein the solidified layer is formulated to deliver the drug at a therapeutically effective rate for at least about 2 hours.

101. A solidified layer as in claim 93, wherein the solidified layer is formulated to deliver the drug at a therapeutically effective rate for at least about 4 hours.

102. A solidified layer as in claim 93, wherein the formulation is formulated to deliver the drug at a therapeutically effective rate for at least about 12 hours.

103. A solidified layer as in claim 93, wherein the solidified layer is a soft, coherent solid that is peelable from a skin surface as a single piece or as only a few large pieces relative to the application size.

104. A solidified layer as in claim 93, wherein the solidified layer is soluble in water.

105. A solidified layer as in claim 93, wherein the solidified layer can be removed by washing.

106. A solidified layer as in claim 93, wherein the solidified layer is flux-enabling for the drug.

107. A solidified layer as in claim 93, wherein the solidified layer is adhesive to the skin surface on a first major surface, and is non-adhesive on an opposing major surface.

108. A solidified layer as in claim 93, wherein the solidified layer is formulated to deliver a majority the drug that is dermally deliverable therefrom while the solidified layer is substantially devoid of water and any solvent more volatile than water.

109. A formulation for treating a subject suffering from alopecia, comprising:
a) a drug including a member selected from the group consisting of clobetasol propionate, clobetasol, derivatives thereof, and combinations thereof;
b) a solvent vehicle, comprising:
i) a volatile solvent system including at least one volatile solvent, and
ii) a non-volatile solvent system comprises at least one solvent selected from the group consisting of propylene glycol, glycerol, and combinations thereof, and at least one solvent selected from the group consisting of isostearic acid, oleic acid, and combinations thereof;
c) a solidifying agent selected from the group consisting of polyvinyl alcohol, fish gelatin, gluten, casein, zein, and combinations thereof;
wherein the formulation has a viscosity suitable for application and adhesion to a skin surface prior to evaporation of the volatile solvent system, wherein the formulation, after being applied to a skin surface as a layer, forms a solidified, coherent, flexible, and continuous layer after at least partial evaporation of the volatile solvent system, and wherein the drug continues to be topically delivered at the therapeutically effective rate after the volatile solvent system is at least substantially all evaporated.

110. A method for treating alopecia, comprising:
a) applying to a skin area of a subject suffering from alopecia a 0.01 mm to 2 mm thick layer of an adhesive solidifying formulation, the formulation comprising
i) a drug including at least one member selected from the group consisting of clobetasol propionate, clobetasol, and combinations thereof;
ii) volatile solvent system including at least one volatile solvent,
iii) a non-volatile solvent system including at least one solvent selected from the group consisting of propylene glycol, glycerol, and combinations thereof, and including at least one solvent selected from the group consisting of isostearic acid, oleic acid, and combinations thereof;
iv) a solidifying agent including at least one member selected from the group consisting of polyvinyl alcohol, fish gelatin, gluten, casein, zein, and combinations thereof;
wherein the formulation has a viscosity suitable for application and adhesion to the palm skin surface prior to evaporation of the volatile solvent system, wherein the formulation layer forms a solidified, coherent and flexible layer after at least partial evaporation of the volatile solvent system, and wherein the drug continues to be topically delivered at the therapeutically effective rate for at least two after the volatile solvent system is at least substantially all evaporated;
b) leaving the formulation on the skin surface for an intended application period of at least 2 hours, and
c) removing the solidified, coherent and flexible layer from the skin surface after the intended application period.
